(19) United States
(12) Patent Application Publication
(10) Pub. No.: US 2016/0058912 A1
(43) Pub. Date: Mar. 3, 2016

(54) SCAFFOLDS FOR PROMOTING CALCIFIED CARTILAGE AND/OR BONE FORMATION

(71) Applicant: THE TRUSTEE OF COLUMBIA UNIVERSITY IN THE CITY OF NEW YORK, New York, NY (US)

(72) Inventors: Helen H. LU, New York, NY (US); Margaret K BOUSHELL, Sandy Hook, CT (US)

(73) Assignee: The Trustees of Columbia University in the City of New York, New York, NY (US)

(21) Appl. No.: 14/784,019

(22) PCT Filed: Apr. 11, 2014
(86) PCT No.: PCT/US14/33843

§ 371 Date: Oct. 12, 2015

Related U.S. Application Data
(60) Provisional application No. 61/811,355, filed on Apr. 12, 2013.

(51) Int. Cl. A61L 27/46; A61L 27/36; C12N 5/077; A61L 27/52

(52) U.S. Cl. CPC A61L 27/46; A61L 27/52; A61L 27/3608; C12N 5/0655; A61L 2430/02; A61L 2430/06

(57) ABSTRACT

Biomimetic hydrogel and selected ceramic interface scaffolds useful in regenerating calcified cartilage and promoting stable and integrative cartilage repair are provided. An aspect of this application relates to scaffolds for promoting calcified cartilage and/or bone formation. The scaffolds of this application comprise a biomimetic hydrogel and a ceramic structure or mineral source selected to modulate biosynthesis and mineralization of chondrocytes.

This patent application claims the benefit of priority from U.S. Provisional Application Ser. No. 61/811,355, filed Apr. 12, 2013, the content of which is herein incorporated by reference in its entirety.

### STATEMENT REGARDING FEDERALLY SPONSORED RESEARCH OR DEVELOPMENT

This invention was made with government support under Grant Number 5R01AR55280 awarded by the National Institutes of Health-National Institute of Arthritis and Musculoskeletal and Skin Diseases (NIH-NIAMS). The government has certain rights in the invention.

### FIELD

The disclosed subject matter relates to biomimetic hydrogel scaffolds for promoting stable and integrative cartilage repair and methods for modulating chondrocyte biosynthesis and mineralization, enhancing matrix production by chrondrocytes or cells capable of chondrogenesis, and forming endochondral and/or osteochondral ossification mediated bone with these scaffolds.

### BACKGROUND

Osteoarthritis is a painful joint condition characterized by cartilage degeneration. This disease currently affects over 27 million adults in the United States and is a leading cause of disability among older Americans. Once damaged, cartilage has a limited capacity for self-repair due to its avascular nature. As a result, surgical intervention is often required. Current treatment strategies are limited, however, by fibrocartilage formation in the defect site and poor graft integration over time.

Osteochondral grafts have emerged as an alternative to surgery. While these grafts show promise for regenerating both cartilage and bone-like tissues, the clinical challenge which remains is the consistent formation of a stable osteochondral interface between these tissues.

### SUMMARY

An aspect of this application relates to scaffolds for promoting calcified cartilage and/or bone formation. The scaffolds of this application comprise a biomimetic hydrogel and a ceramic structure or mineral source selected to modulate biosynthesis and mineralization of chondrocytes.

Another aspect of this application relates to a method for modulating chondrocyte biosynthesis and mineralization in a hydrogel. The method comprises adding to a biomimetic hydrogel a ceramic structure or mineral source selected to modulate biosynthesis and mineralization of chondrocytes.

Another aspect of this application relates to a method for enhancing matrix production by chrondrocytes or cells capable of chondrogenesis. The method comprises culturing the cells on a scaffold comprising a biomimetic hydrogel and ceramic structure or mineral source selected to modulate biosynthesis and mineralization of chondrocytes.

Yet another aspect of this application relates to forming endochondral and/or osteochondral ossification mediated bone with these scaffolds.

### BRIEF DESCRIPTION OF THE FIGURES

FIGS. 1A through 1C shows results of characterization of a CDA and TCP ceramic powder prior to scaffold fabrication via scanning electron microscope (SEM; FIG. 1A), X-ray diffraction (XRD; FIG. 1B) and Fourier transfer infrared spectroscopy (FTIR; FIG. 1C).

FIGS. 2A and 2B show results of chondrocyte proliferation and distribution analysis in CDA, TCP and CaP-free (ceramic free, hydrogel only) scaffolds. FIG. 2A shows cell number at days 1, 7 and 14 while FIG. 2B provides results of hematoxylin and eosin staining at day 14.

FIGS. 3A and 3B show results of assessment of GAG content in the matrix deposition. FIG. 3A is a bargraph comparing levels of GAG as a percentage of wet weight of the scaffold at days 1, 7 and 14 in CDA, TCP and CaP-free scaffolds. FIG. 3B compares Alcian Blue staining at day 14 in CDA, TCP and CaP-free scaffolds.

FIGS. 4A and 4B show results of assessment of collagen content in the matrix deposition. FIG. 4A is a bargraph comparing levels of collagen as a percentage of wet weight of the scaffold at days 1, 7 and 14 in CDA, TCP and CaP-free scaffolds. FIG. 4B compares levels of collagen I-V, collagen I and collagen II at day 14 in CDA, TCP and CaP-free scaffolds.

FIGS. 5A and 5B shows results of the mineralization potential analyzed by measuring the ALP activity of scaffolds further containing CDA or TCP as well as CaP-free scaffolds at days 1, 7 and 14 (see FIG. 5A) and the calcium content measured by Alizarin Red staining at days 1 and 14 (see FIG. 5B).

FIGS. 6A through 6C show the effects of scaffolds further containing CDA or TCP as well as CaP-free scaffolds on hypertrophic markers including collagen X at day 14 (FIG. 6A) as well as collagen X, Indian hedgehog (Ihh) and matrix metalloproteinase 13 (MMP13) at days 1 (FIG. 6B) and 14 (FIG. 6C).

FIGS. 7A and 7B show the effects of scaffolds further containing CDA and TCP as well as CaP-free scaffolds on media ion concentrations of Ca⁺² and PO₄⁻³.

### DETAILED DESCRIPTION

### Definitions

In order to facilitate an understanding of the material which follows, one may refer to Freshney, R. Ian. Culture of Animal Cells—A Manual of Basic Technique (New York: Wiley-Liss, 2000) for certain frequently occurring methodologies and/or terms which are described therein.

Unless defined otherwise, all technical and scientific terms used herein have the meaning commonly understood by a person skilled in the art to which this invention belongs. However, except as otherwise expressly provided herein, each of the following terms, as used in this application, shall have the meaning set forth below.

As used herein, “ALP activity” shall mean alkaline phosphatase activity.

As used herein, a “biocompatible” material is a synthetic or natural material used to replace part of a living system or to function in intimate contact with living tissue. Biocompatible materials are intended to interface with biological systems to evaluate, treat, augment or replace any tissue, organ or function of the body. The biocompatible material has the ability to perform with an appropriate host response in a specific application and does not have toxic or injurious effects on biological systems. Nonlimiting examples of biocompatible materials include a biocompatible ceramic, a biocompatible mineral source, a biocompatible polymer or a biocompatible hydrogel.

As used herein, “biodegradable” means that the material, once implanted into a host, will begin to degrade.

As used herein, “biomimetic” shall mean a resemblance of a synthesized material to a substance that occurs naturally in a human body and which is not substantially rejected by (e.g., does not cause an unacceptable adverse reaction in) the human body. When used in connection with the tissue scaffolds, biomimetic means that the scaffold is substantially biologically inert (i.e., will not cause an unacceptable immune response/rejection) and is designed to resemble a structure (e.g., soft tissue anatomy) that occurs naturally in a mammalian, e.g., human, body and that promotes healing when implanted into the body.

As used herein, “chondrocyte” shall mean a differentiated cell responsible for secretion of extracellular matrix of cartilage.

As used herein, “chondrogenesis” shall mean the formation of cartilage tissue.

As used herein, “effective amount” shall mean a concentration, combination or ratio of one or more components added to the scaffold which promote growth and/or proliferation or cells and/or direct differentiation of stem cells to a selected cell type. Such components may include, but are not limited to, ceramic structures, mineral sources one or more extracellular matrix components, physical or mechanical stimulation and chemical stimulation such as media or growth factors which promote growth and/or proliferation or cells and/or direct differentiation of stem cells to a selected cell type.

As used herein, “hydrogel” shall mean any colloid in which the particles are in the external or dispersion phase and water is in the internal or dispersed phase.

As used herein, “polymer” means a chemical compound or mixture of compounds formed by polymerization and including repeating structural units. Polymers may be constructed in multiple forms and compositions or combinations of compositions.

By “osteochondral interface” it is meant a region composed of hypertrophic chondrocytes in a mineralized matrix. This interfacial zone is important because it serves to anchor the articular cartilage to the subchondral bone and allows for pressurization of articular cartilage during loading. This region also limits vascular invasion of the articular cartilage.

As used herein, “stem cell” means any unspecialized cell that has the potential to develop into many different cell types in the body, such as chondrocytes or chondrocyte progenitor cells. Nonlimiting examples of “stem cells” include mesenchymal stem cells, embryonic stem cells and induced pluripotent cells.

As used herein, “synthetic” shall mean that the material is not of a human or animal origin.

As used herein, all numerical ranges provided are intended to expressly include at least the endpoints and all numbers that fall between the endpoints of ranges.

The following embodiments are provided to further illustrate the methods of tissue scaffold production of this application. These embodiments are illustrative only and are not intended to limit the scope of this application in any way.

### EMBODIMENTS

The disclosed subject matter relates to scaffolds comprising a biomimetic hydrogel and a ceramic structure or mineral source selected to modulate biosynthesis and mineralization of chondrocytes and methods for use of these scaffolds in modulating chondrocyte biosynthesis and mineralization, enhancing matrix production by chrondrocytes or cells capable of chondrogenesis and/or forming endochondral and/or osteochondral ossification mediated bone. In one embodiment, the ceramic structure or mineral source is selected to modulate biosynthesis and mineralization of deep zone chondrocytes and/or hypertrophic chondrocytes.

It is expected that any polymer chain hydrogel useful as a tissue scaffold can be used. Examples include, but are not limited to, agarose, carrageenan, polyethylene oxide, polyethylene glycol, tetraethylene glycol, triethylene glycol, trimethylolpropane ethoxylate, pentaerythritol ethoxylate, hyaluronic acid, thiosulfonate polymer derivatives, polyvinylpyrrolidone-polyethylene glycol-agar, collagen, dextran, heparin, hydroxyalkyl cellulose, chondroitin sulfate, dermatan sulfate, heparan sulfate, keratan sulfate, dextran sulfate, pentosan polysulfate, chitosan, alginates, pectins, agars, glucomannans, galactomannans, maltodextrin, amylose, polyalditol, alginate-based gels cross-linked with calcium, polymeric chains of methoxypoly(ethylene glycol) monomethacrylate, chitin, poly(hydroxyalkyl methacrylate), poly(electrolyte complexes), poly(vinylacetate) cross-linked with hydrolysable bonds, water-swellable N-vinyl lactams, carbomer resins, starch graft copolymers, acrylate polymers, polyacrylamides, polyacrylic acid, ester cross-linked polyglucans, and derivatives and combinations thereof.

In one embodiment, the hydrogel used in the scaffolds of this application is agarose. Agarose offers a controlled inert matrix which supports a rounded chondrocyte phenotype. Furthermore, agarose allows for accumulation and retention of matrix products.

Scaffolds of this application further comprise a ceramic structure or alternative mineral source selected to modulate biosynthesis and mineralization of chondrocytes. Nonlimiting examples of ceramic structures or mineral sources which can be used include, bioactive glasses, hydroxyapatite, calcium deficient apatite, biphasic calcium phosphate, and combinations of these. In one embodiment the ceramic structure is a calcium phosphate. A nonlimiting example of a calcium phosphate ceramic is beta-tricalcium phosphate (TCP). As will be understood by the skill artisan upon reading this disclosure, however, alternative ceramic structures and mineral sources can be selected. Further, as will also be understood by the skilled artisan upon reading this disclosure, parameters including, but not limited to, chemistry, crystallinity and/or particle size of the ceramic structure or mineral source can be selected to modulate and/or direct chondrocyte response. In one embodiment, the selected ceramic structure is calcium-deficient apatite (CDA).

In one embodiment, a ceramic structure is added at a concentration ranging from about 0.5% to 4.5% of the ceramic.

In one embodiment, the concentration of ceramic structure or mineral source added is selected to mimic physiologic levels or superphysiologic levels of the selected cell type and selected ceramic.

Without being bound to any particular theory, it is believed that the ceramic structure and/or mineral source will serve as part of any bone or calcified cartilage formed on the scaffold.

The tissue engineered scaffolds may further comprise chondrogenic cells or chondrocytes. By “chondrogenic cells” is it meant to include any cell capable of chrondrogenic differentiation. In one embodiment, the tissue engineered scaffold is seeded with mesenchymal stem cells. In one embodiment, the mesenchymal stem cells are human mesenchymal stem cells. Examples of alternative cells for seeding include, but are not limited to, adipose derived stem cells, synovium derived stem cells, induced pluripotent stem cells, embryonic stem cells, and fibrochondrocytes.

Scaffolds of this application comprising the biomimetic hydrogel agarose and the ceramic structures TCP and CDA, selected to modulate biosynthesis and mineralization of chondrocytes were fabricated. CaP-free agarose scaffolds were also fabricated as a control.

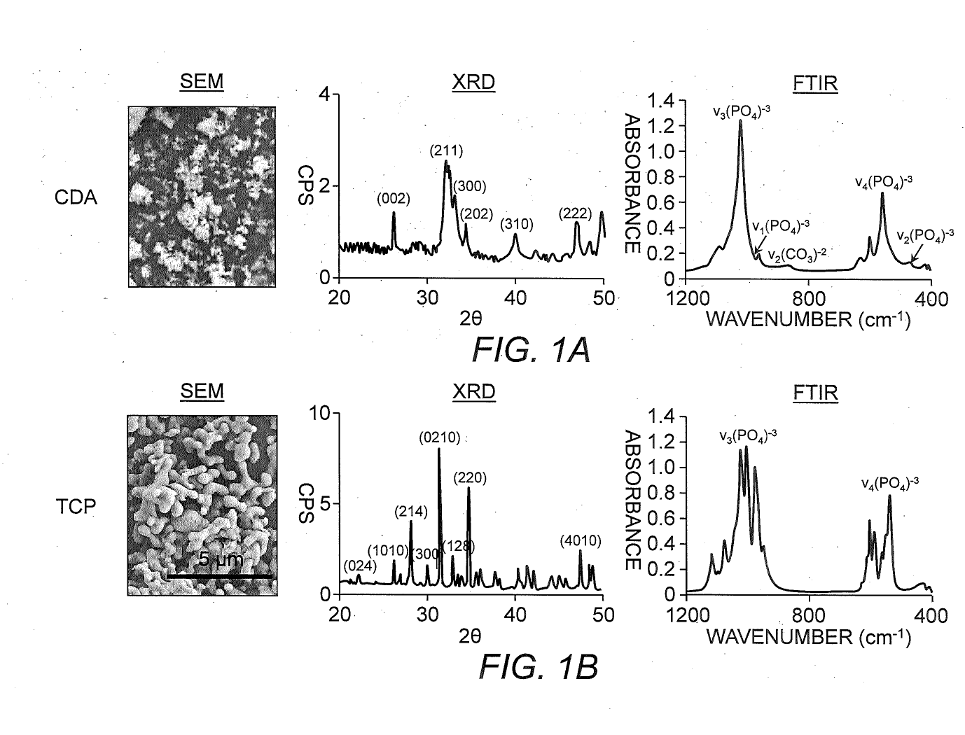

Before scaffold fabrication, the ceramic powders were characterized via SEM, XRD (see FIG. 1B), FTIR (FIG. 1C), and ICP (see Table 1). From the SEM imaging depicted in FIG. 1A, it was clear that the TCP powder had a clearly defined, rhombic particle shape while the CDA powder did not. XRD spectra depicted in FIG. 1B showed broad peaks for the CDA powder, indicative of small crystallite size and poor crystallinity, whereas TCP resulted in sharp peaks matching the spectra for TCP powder. FTIR depicted in FIG. 1C showed the presence of carbonate in the CDA powder whereas the TCP powder was carbonate free and marked by split phosphate bending curves. ICP analysis, results of which are depicted in Table 1, showed no difference in calcium phosphate ratio between the two powders.

TABLE 1
Results of ICP Analysis

| | | | Ca/P Molar |
|---|---|---|---|
| | Ca (wt %) | P (wt %) | Ratio |
| CDA (n = 6) | 31.58 ± 0.36 | 17.34 ± 0.14 | 1.41 ± 0.02 |
| TCP (n = 6) | 33.54 ± 0.40 | 18.56 ± 0.18 | 1.40 ± 0.02 |

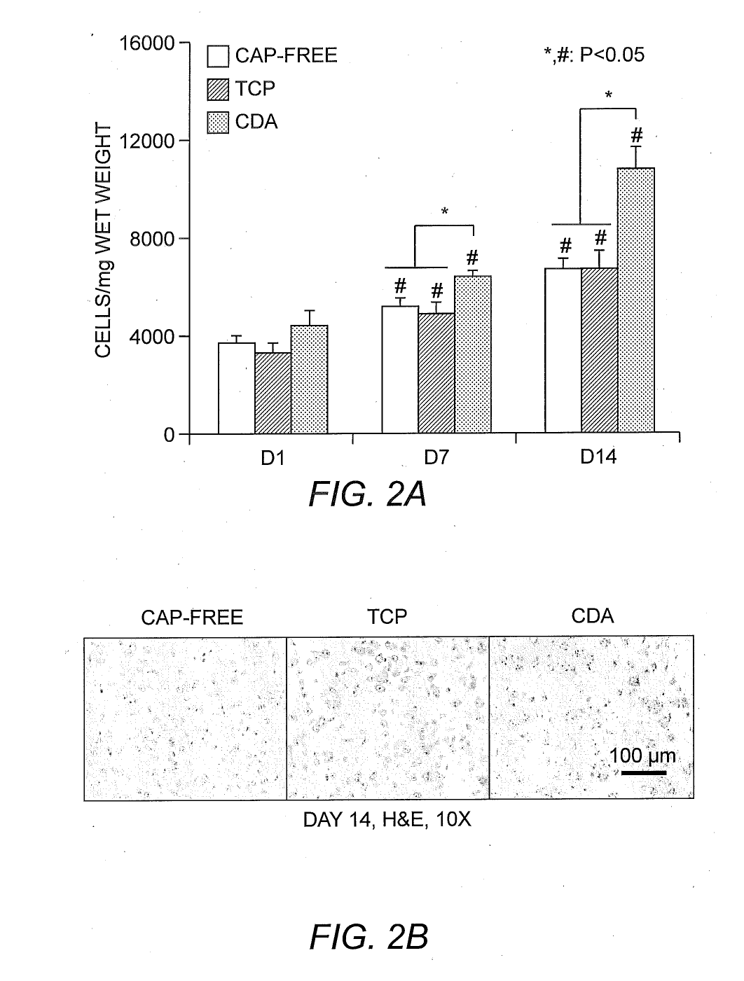

After acellular characterization was completed, cell viability and distribution of cells seeded on hydrogel scaffolds further containing CDA or TCP was assessed. See FIGS. 2A and 2B. Cell number was determined at days 1, 7 and 14. Cell number increased on scaffolds further containing CDA or TCP as well as CaP-free scaffolds. However, significantly higher number of cells were observed on days 7 and 14 for the scaffold further containing CDA (see FIG. 2A).

Once it was determined that the cells were viable in the scaffolds, matrix deposition was assessed by looking at both collagen and GAG content.

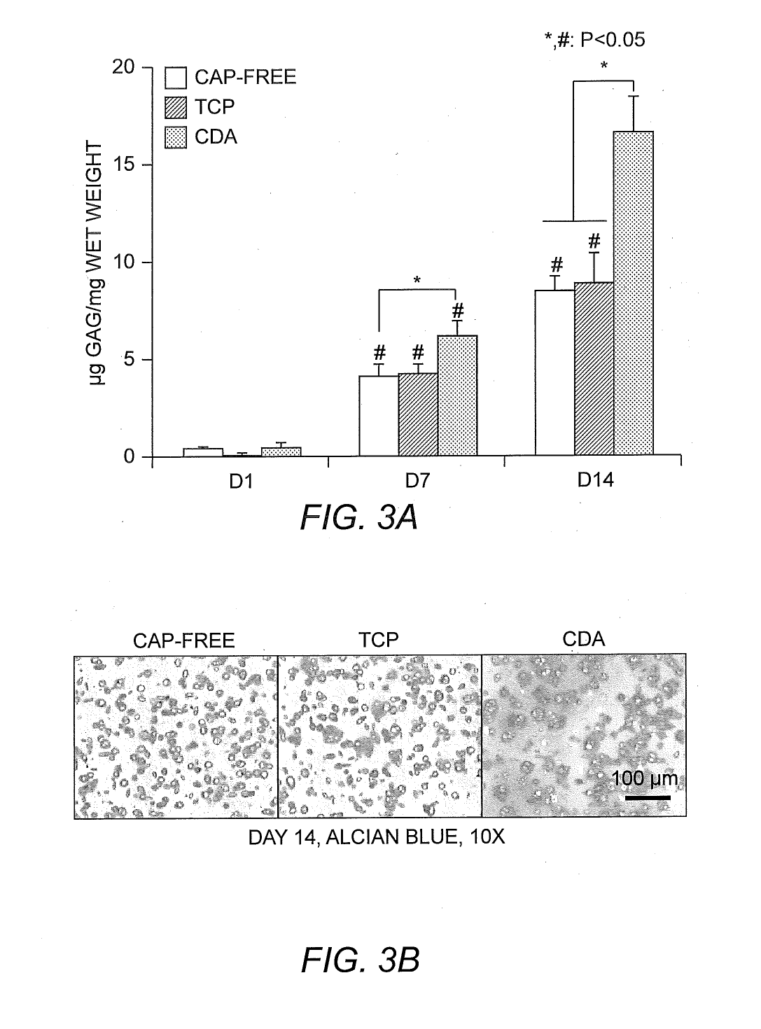

Results for GAG content are shown in FIGS. 3A and 3B. As shown in FIGS. 3A and 3B, GAG content increased in scaffolds further containing CDA or TCP as well as CaP-free scaffolds and no differences in GAG deposition were observed between the CaP-free and TCP containing scaffolds. The highest GAG deposition observed was in the CDA containing scaffold on day 14.

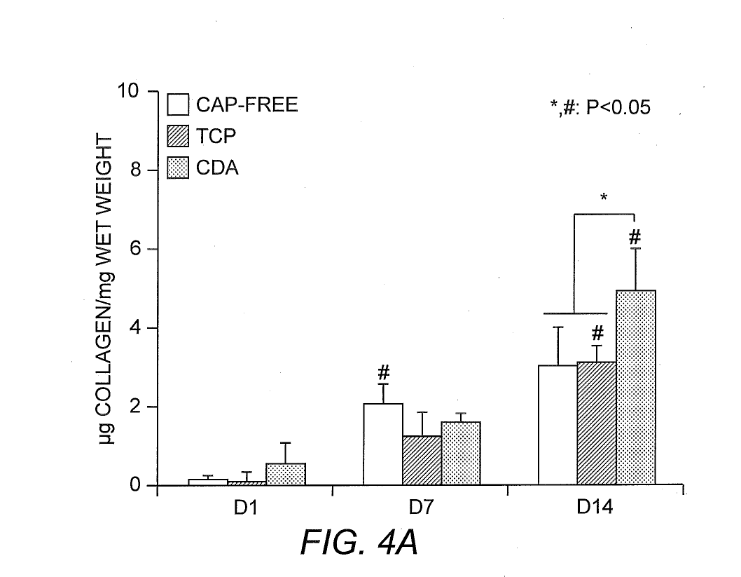

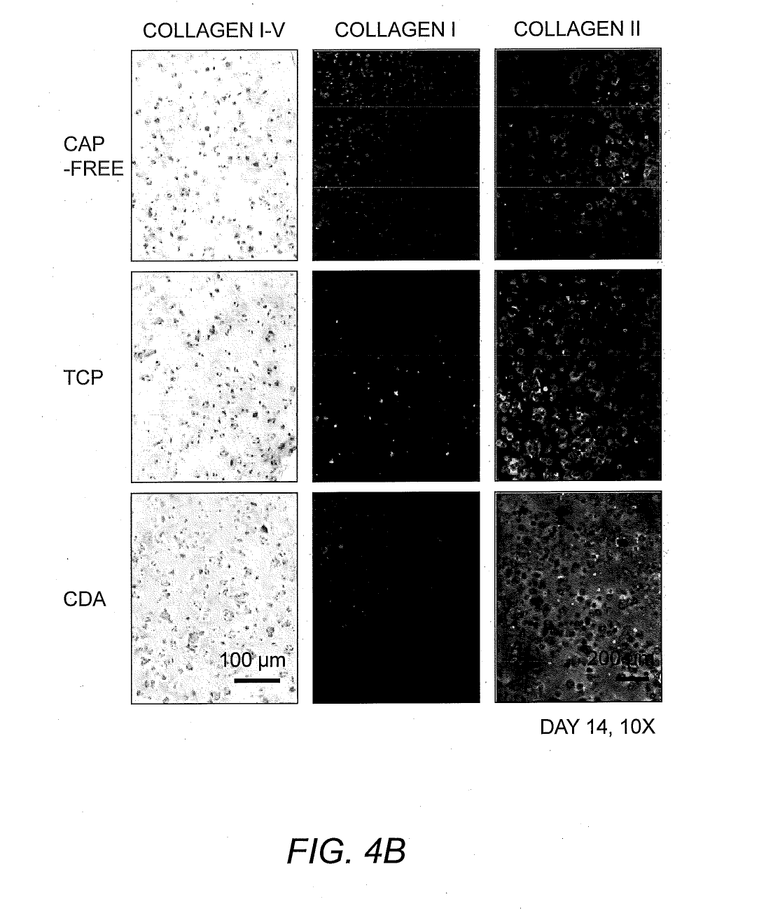

Results for collagen content are shown in FIGS. 4A and 4B. As shown in FIGS. 4A and 4B, collagen content increased in scaffolds further containing CDA or TCP as well as CaP-free scaffolds and no differences in collagen deposition were observed between the CaP-free and TCP containing scaffolds. The highest collagen deposition observed was in the CDA containing scaffold on day 14 which tested positive for collagen II.

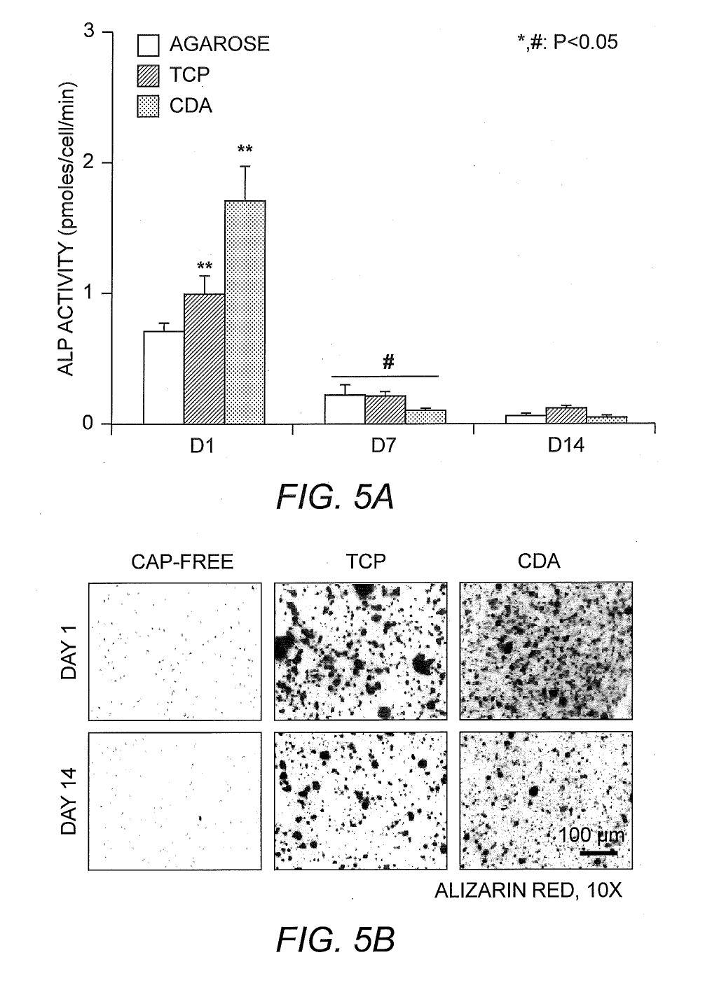

Mineralization potential was analyzed by measuring the ALP activity of scaffolds further containing CDA or TCP as well as CaP-free scaffolds at days 1, 7 and 14 (see FIG. 5A) and the calcium content at days 1 and 14 (see FIG. 5B). ALP activity decreased in all scaffolds (see FIG. 5A) while only scaffolds containing a ceramic structure were positive for calcium via Alizarin staining (see FIG. 5B).

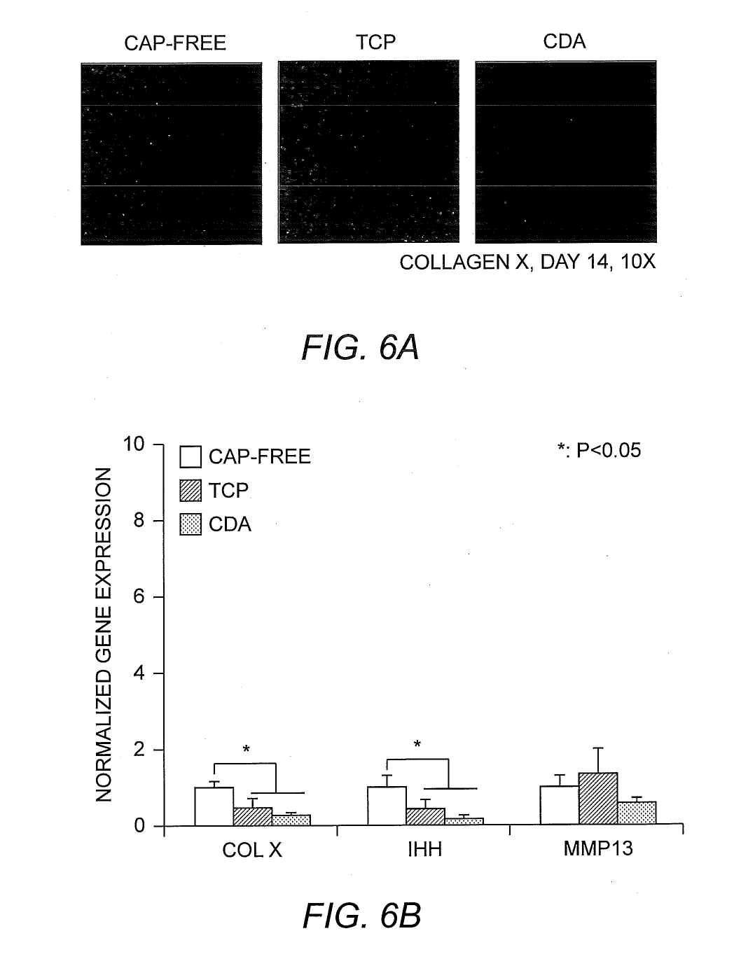

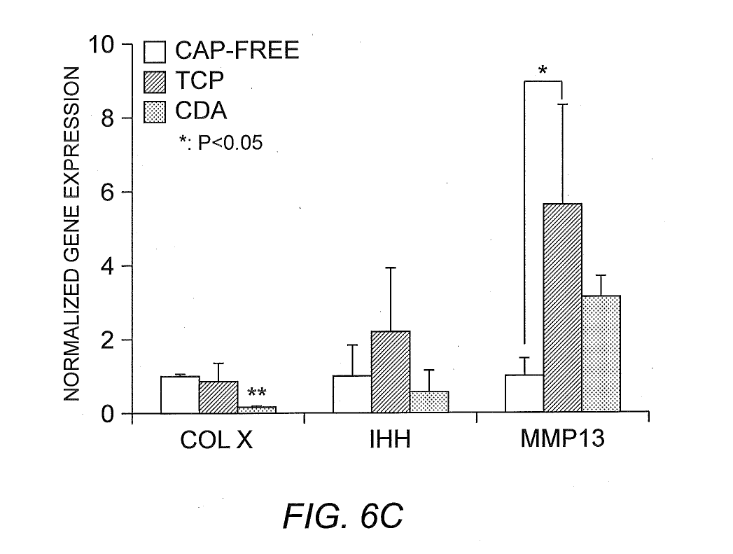

The effect of scaffolds further containing CDA or TCP on hypertrophic markers collagen X, Ihh and MMP13 was also examined. Results are shown in FIGS. 6A through 6C. As shown in FIG. 6B, hypertrophic markers were downregulated at day 1 in all the scaffolds tested. Further, collagen X was lowest in scaffolds further containing CDA at day 14 (see FIGS. 6A and 6C). However, as shown in FIG. 6C, MMP13 was upregulated in scaffolds containing TCP at day 14.

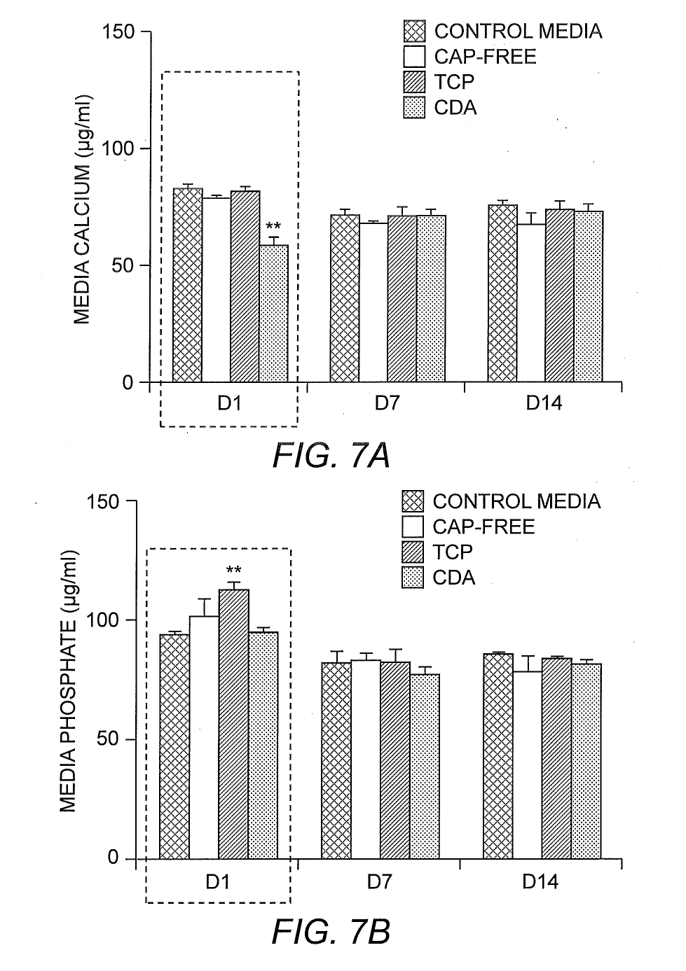

In addition to cell biosynthesis and mineralization, the media was analyzed to determine relevant changes in media ion concentrations of Ca²⁺ and PO₄³⁻. Results are shown in FIGS. 7A and 7B. On day 1, Ca²⁺ media concentrations were decreased in scaffolds further containing CDA (see FIG. 7A) while PO₄³⁻ concentrations were increased in scaffolds further containing TCP (see FIG. 7B). After day 1, however, there were no differences in ion concentration in media between the scaffolds.

These results show that ceramic type modulated chondrocyte response. While not being bound to any particular theory, it is believed that the change in cell response may be due to parameters which were altered during sintering. Table 2 shows ceramic characteristics of CDA and TCP following sintering.

TABLE 2
CDA vs. TCP Ceramics

| | Particle | Crystallite | | Ca/P Molar |
|---|---|---|---|---|
| | Shape | Size | Chemistry | Ratio |
| CDA | Irregular | Small | Carbonated | 1.41 |
| TCP | Rhombic | Large | Carbonate- | 1.40 |
| | | | free | |

For instance, the particle shape was changed from irregular to rhombic during the sintering and the crystallite size increased from small to large which was detected by decreasing peak width in the XRD spectra. Finally the carbonate which was present in the CDA was removed during sintering. Both ceramic types had the same Ca/P molar ratio.

Also shown by these experiments is that CDA promoted cell proliferation and enhanced matrix deposition while TCP did not. Further, while ALP activity decreased over time in all the scaffolds, only CDA downregulated hypertrophic markers.

Similar experiments were conducted comparing scaffolds of this application stimulated by addition of triiodothyronine or thyroid hormone, also referred to as T3. This hormone stimulates the hypertrophic phenotype in deep zone chondrocytes. T3 (25 nM) was added in the day 1 and day 3 feeding with no subsequent treatment after that. The CDA containing scaffold in the presence and absence of T3 stimulation measured the highest cell number at day 14. Hematoxylin and eosin staining revealed uniform cell distribution throughout all the T3 stimulated as well as unstimulated scaffolds at day 14. The CDA scaffolds also measured the highest collagen deposition on day 14 in the presence and absence of T3 stimulation. Picrosirius red staining showed positive collagen staining throughout all the scaffolds with the most positive staining observed for CDA containing scaffolds on day 14 in the presence and absence of T3 stimulation. The CDA scaffolds also measured the highest proteoglycan deposition on day 14 in the presence and absence of T3 stimulation. Alcian blue staining showed positive GAG staining throughout all the scaffolds with the most positive staining observed for CDA containing scaffolds on day 14 in the presence and absence of T3 stimulation. The CDA scaffolds also measured the highest ALP activity on day 1 with low ALP activity thereafter in the presence and absence of T3. Finally, TCP and CDA containing scaffolds exhibited downregulated Col X and Ihh on day 1. The CDA containing scaffolds also exhibited the lowest ALP expression and the highest PTHrP. On day 14, the untreated CDA containing scaffold exhibited the lowest Col X, Ihh, and ALP, although there were no significant differences between scaffolds for these genes. MMP13 was upregulated at day 14 for the unstimulated TCP containing scaffold. A T3-stimulated HA containing scaffold exhibited upregulated Ihh on day 14. All stimulated groups exhibited decreased MMP13 expression with respect to the stimulated control group.

Accordingly, as shown by these experiments, crystal structure, ceramic chemistry and particle size are critical parameters for calcified cartilage scaffold design. As further shown, selection of the ceramic structure or mineral source to be added to a biomimetic hydrogel can modulate biosynthesis and mineralization of chondrocytes and enhance matrix production by chrondrocytes or cells capable of chondrogenesis. As also shown by these experiments, a preferred embodiment of the present invention is a biomimetic hydrogel scaffold further containing CDA.

In one embodiment, a stimulant such as T3 is added to the scaffold.

The tissue engineered scaffolds of this application are useful in studying chrondrogenesis, promoting proliferation and chondrogenesis of chondrogenic cells and producing functional cartilage.

These scaffolds can be used in combination with a cartilage graft to promote functional integration at the cartilage-bone interface. This can be done by layering this scaffold with a mineral free cartilage scaffold or an allo- and autograft. Cartilage scaffolds can be made from a variety of hydrogels, including, but not limited to alginate, PEG, chitosan and hyaluronic acid.

Biomimetic hydrogel and selected ceramic or mineral interface scaffolds of this application are useful in regenerating calcified cartilage, promoting stable and integrative cartilage repair and for osteochondral ossification mediated and/or endochondral bone formation.

The disclosed subject matter is further illustrated by the following nonlimiting example.

The following disclosure should not be construed as limiting the invention in any way. One of skill in the art will appreciate that numerous modifications, combinations, rearrangements, etc. are possible without exceeding the scope of the invention. While this invention has been described with an emphasis upon various embodiments, it will be understood by those of ordinary skill in the art that variations of the disclosed embodiments can be used, and that it is intended that the invention can be practiced otherwise than as specifically described herein.

### EXAMPLES

### Example 1

### Scaffold Production

The chondrocytes were encapsulated at a density of 10 million cells/ml in sterile 2% low gelling agarose (Agarose Type VII, Sigma, St. Louis, Mo.) and a biopsy punch (Sklar Instruments, West Chester, Pa.) was used to core cylindrical scaffolds (θ=5 mm, height=2.4 mm). Acellular and cellular agarose scaffolds with 1.5 w/v % ceramic (Sigma) and corresponding samples without ceramic were fabricated. All samples were cultured under humidified conditions at 37° C. and 5% CO₂, and maintained in ITS culture medium composed of DMEM supplemented with 1% ITS+ Premix (BD Biosciences, San Jose, Calif.), 1% penicillin-streptomycin, 0.1% gentamicin sulfate, 0.1% antifungal, and 40 μg/ml L-proline (Sigma). The medium was changed every other day and freshly supplemented with 50 μg/mL ascorbic acid (Sigma). The responses of deep zone chondrocytes were compared in CDA-1, CDA-2, and ceramic-free scaffolds over a two-week culture period.

### Example 2

### Methods Used to Characterize Ceramic Powder

Ceramic particle shape was assessed using scanning electron microscopy (SEM, Hitachi 4700 FE-SEM, 5 kV, 1000×). Particles were sputter-coated with gold for 20 seconds before SEM imaging (Cressington 108 Auto, Watford, UK). Ceramic calcium and phosphorus content was determined using inductively coupled plasma analysis (ICP, Thermo Jarrell Ash, Trace Scan Advantage). Briefly, 10 mg of ceramic was dissolved in several drops of 17% HCl and brought to 100 ml with double distilled water. The resulting solutions were pumped through argon plasma excited by a 2 kW/27.12 MHz radiofrequency generator. The concentrations of each element were determined using their characteristic wavelengths (Ca, 317.9 Å; P, 213.6 Å). The crystal structure of the ceramics was evaluated with X-ray diffraction (XRD, X-ray Diffractometer, Inel, Artenay, France). The samples were evaluated over a range of 0-120°, with a step size of 0.029°. Ceramic chemistry was examined using Fourier transform infrared spectroscopy (FTIR, FTS 3000MX Excalibur Series, Digilab, Randolph, Mass.), wherein the samples were dehydrated, mixed with potassium bromide and FTIR spectra were collected in absorbance mode (400 scans, 4 cm⁻¹ resolution).

### Example 3

### Cell Proliferation and Distribution Analysis

Cell proliferation (n=5) was determined using the Quanti-it™ PICOGREEN dsDNA assay kit (Molecular Probes, Eugene, Oreg.) following sample digestion. Briefly, the samples were exposed to a freeze-thaw cycle in 500 μl of 0.1% Triton-X solution (Sigma) in order to lyse the cells. The samples were desiccated for 12 hours (CentriVap Concentrator, Labconco Co., Kansas City, Mo.) and digested with papain (8.3 activity units/ml) in 0.1M sodium acetate (Sigma), 10 mM cysteine HCl (Sigma), and 50 mM ethylenediaminetetraacetate (Sigma) at 65° C. for 18 hours. 25 μl aliquot of the sample was mixed with 175 μl of the PICOGREEN working solution and fluorescence was measured with a microplate reader (Tecan, Research Triangle Park, N.C.), at the excitation and emission wavelengths of 485 and 535 nm, respectively. The conversion factor of 7.7 pg DNA/cell was used to determine cell number.

### Example 4

### Assessment of GAG Content

Sample glycosaminogylcan content (GAG, n=5) was determined with a modified 1,9-dimethylmethylene blue (DMMB) binding assay, with chondrotin-6-sulfate (Sigma-Aldrich, St. Louis, Mo.) as the standard. Briefly, a 10 μl aliquot of sample was diluted (1:4) with dH₂O and mixed with 250 μl of DMMB dye. The absorbance difference between 540 nm and 595 nm was used to improve the sensitivity in signal detection. Alician Blue histology staining was used to qualitatively assess proteoglycan distribution (n=2).

### Example 5

### Assessment of Collagen Content

Total collagen content (n=5) was quantified using a modified hydroxyproline assay with a bovine collagen type I solution (Biocolor, UK) as a standard. All matrix values were normalized by sample wet weight in order to account for any differences in sample size. Additionally, collagen distribution (n=2) was evaluated via histology. Briefly, dehydrated samples were embedded in paraffin (Paraplast X-tra Tissue Embedding Medium, Fisher Scientific) and 7 μm sections were obtained from the center of the scaffold. Total collagen distribution was visualized using Picrosirius Red staining (n=2) while collagen types I and II were assessed using immunohistochemistry. Specifically, monoclonal antibodies for collagen type I (1:00 dilution) and collagen type II (1:100 dilution) were purchased from Abcam (Cambridge, Mass.). After fixation, samples were treated with 1% hyaluronidase for 30 minutes at 37° C. and incubated with primary antibody overnight. All samples were counterstained with DAPI (Sigma). A FITC-conjugated secondary antibody (1:200 dilution, LSAB2 Abcam) was used and samples were imaged under confocal microscopy (Olympus Fluoview IX70) at excitation and emission wavelengths of 488 nm and 568 nm, respectively.

### Example 6

### Mineralization Potential Analysis

Alkaline phosphatase (ALP) activity (n=5) was measured using an colorimetric assay based on the hydrolysis of p-nitrophenyl phosphate (pNP-PO₄) to p-nitrophenol (pNP). Briefly, the samples were lysed in 0.1% TRITON X solution, exposed to a freeze-thaw cycle, and crushed with a mortar. A 25 μl aliquot was added to pNP-PO₄ solution (Sigma) and incubated for 10 min at 37° C. Absorbance was measured at 405 nm using a microplate reader (Tecan). In addition, calcium distribution (n=2) was evaluated using Alizarin Red staining as an indicator of overall mineral distribution.

### Example 7

### Measurement of Hypertrophic Markers

The expression (n=3) of collagen type X, matrix metalloproteinase-13 (MMP-13), Indian Hedgehog (Ihh), Runt-related transcription factor 2 (Runx 2), and parathyroid hormone-related protein (PTHrP) were measured at day 7 using reverse transcription followed by polymerase chain reaction (RT-PCR). Briefly, total RNA was isolated via TRIzol (Invitrogen, Carlsbad, Calif.) extraction, and then was reverse-transcribed into cDNA using the SuperScript III First-Strand Synthesis System (Invitrogen). The cDNA product was amplified with recombinant Platinum Taq DNA polymerase (Invitrogen). Expression band intensities of relevant genes were analyzed semi-quantitatively and normalized to the housekeeping gene glyceraldehydes 3-phosphate dehydrogenase (GAPDH).

### Example 8

### Measurement of Media Ion Concentrations

Media calcium concentrations (n=5) were quantified using the Arsenazo III dye (Pointe Scientific, Lincoln Park, Mich.), with absorbance measured at 620 nm using a microplate reader. Media aliquots were collected at each feeding and the BioVision Phosphate Assay Kit was used to analyze media phosphate levels. Briefly, media was diluted with water in a 1:10 ratio and allowed to react with 30 ul of dye for 30 minutes. Absorbance was measured at 650 nm using a microplate reader (Tecan).

What is claimed is:

1. A scaffold for promoting calcified cartilage and/or bone formation, said scaffold comprising a biomimetic hydrogel and a ceramic structure or mineral source selected to modulate biosynthesis and mineralization of chondrocytes.

2. The scaffold of claim 1 wherein the ceramic structure or mineral source is selected to modulate biosynthesis and mineralization of deep zone chondrocytes and/or hypertrophic chondrocytes.

3. The scaffold of claim 1 wherein the ceramic structure selected comprises calcium-deficient apatite.

4. The scaffold of claim 1 wherein the hydrogel comprises a polymer chain hydrogel.

5. The scaffold of claim 1 further comprising chrondrocytes or cells capable of chondrogenesis.

6. A method for modulating chondrocyte biosynthesis and mineralization in a hydrogel, said method comprising adding to the hydrogel a ceramic structure or mineral sources selected to modulate biosynthesis and mineralization of chondrocytes.

7. The method of claim 6 wherein the ceramic structure or mineral source is selected to modulate biosynthesis and mineralization of deep zone chondrocytes and/or hypertrophic chondrocytes.

8. The method of claim 6 wherein the ceramic structure selected comprises calcium-deficient apatite.

9. The method of claim 6 wherein the hydrogel comprises a hydrophilic polymer chain hydrogel.

10. The method of claim 6 further comprising adding chrondrocytes or cells capable of chondrogenesis to the hydrogel and ceramic structure.

11. A method for enhancing matrix production by chrondrocytes or cells capable of chondrogenesis, said method comprising culturing the cells on a scaffold comprising a biomimetic hydrogel and a ceramic structure or mineral source selected to modulate biosynthesis and mineralization of chondrocytes.

12. The method of claim 11 wherein the ceramic structure or mineral source is selected to modulate biosynthesis and mineralization of deep zone chondrocytes and/or hypertrophic chondrocytes.

13. The method of claim 11 wherein the ceramic structure selected comprises calcium-deficient apatite.

14. A method for promoting cartilage regeneration and integration at an interface of bone and cartilage in a subject in need thereof, said method comprising transplanting into the subject a scaffold of any of claims 1 through 5.

15. A method for forming endochondral and/or osteochondral ossification mediated bone comprising culturing cells capable of forming the bone on a scaffold comprising a biomimetic hydrogel and a selected ceramic structure or mineral source.

16. The method of claim 15 wherein the ceramic structure selected comprises calcium-deficient apatite.
